(19) United States
(12) Patent Application Publication
(10) Pub. No.: US 2026/0326035 A1
(43) Pub. Date: Oct. 22, 2026

(54) FILTRATION SYSTEMS AND METHODS

(71) Applicant: Repligen Corporation, Waltham, MA (US)

(72) Inventors: Derek Carroll, Los Angeles, CA (US); Steven Cates, Washoe Valley, NV (US); Mark Perreault, Hudson, MA (US); Travis Ward, Stow, MA (US)

(73) Assignee: Repligen Corporation, Waltham, MA (US)

(21) Appl. No.: 19/116,678

(22) PCT Filed: Oct. 9, 2023
(86) PCT No.: PCT/US2023/076344

§ 371 Date: Mar. 28, 2025

Related U.S. Application Data
(60) Provisional application No. 63/378,893, filed on Oct. 10, 2022.

(51) Int. Cl. B01D 61/20; B01D 61/18; B01D 61/22; B01D 63/02; B01D 65/02; C12M 1/00

(52) U.S. Cl. CPC B01D 61/20; B01D 61/18; B01D 61/22; B01D 63/02; B01D 65/02; C12M 47/10; B01D 2311/2688; B01D 2313/18; B01D 2313/502; B01D 2321/04; B01D 2321/2083

(57) ABSTRACT

A fluid filtration system includes a fluid storage vessel such as a bioreactor, one or more filters housings, and at least one pump coupled between the fluid storage vessel and the filter housing(s). A plurality of valves are used to control flow between the pump and the filter housing. The at least one pump is configured to move fluid from the fluid storage vessel through the filter housing(s), and the valves are configured for selectively directing fluid received from the at least one pump to the first or second end of the filter housing(s). A plunger pump can be used to selectively move flow between first and second ends of a filter housing.

### BACKGROUND OF THE DISCLOSURE

### Field of the Disclosure

Embodiments of the disclosure relate generally to filtration systems, and more particularly to filtration systems including one or more single pumps for selectively providing flow of process fluid in two opposing directions through a filter member.

### Discussion of Related Art

Filtration is often performed to separate, clarify, modify, and/or concentrate a fluid solution, mixture, or suspension. In the biotechnology, pharmaceutical, and medical industries, filtration is vital for the successful production, processing, and analysis of drugs, diagnostics, and chemicals as well as many other products. As examples, filtration may be used to sterilize fluids and to clarify a complex suspension into a filtered “clear” fraction and an unfiltered fraction. Similarly, constituents in a suspension may be concentrated by removing or “filtering out” the suspending medium. Further, with appropriate selection of filter material, filter pore size and/or other filter variables, many other specialized uses have been developed. These uses may involve selective isolation of constituents from various sources, including cultures of microorganisms, blood, as well as other fluids that may be solutions, mixtures, or suspensions.

Biologics manufacturing processes have advanced through substantial process intensification. Both eukaryotic and microbial cell culture to produce recombinant proteins, virus-like particles (VLP), gene therapy particles, and vaccines now include cell growth techniques that can achieve 100e6 cells/ml or higher. This is achieved using cell retention devices that remove metabolic waste products and refresh the culture with additional nutrients. One common means of cell retention is to perfuse a bioreactor culture using hollow fiber filtration using alternating tangential flow (ATF).

Commercial and development scale processes use a device that controls a pump to perform ATF through a hollow fiber filter. Typically, these systems are arranged so that the pump moves fluid through the filter in a single direction. This results in the culture spending an undesirably long time outside the bioreactor and can also cause premature fouling of the hollow fiber filter.

It would be desirable, therefore, to provide an improved pumping arrangement that increases the utilization of the entire filter length of a hollow fiber filter used in connection with a vessel such as a bioreactor vessel. It would also be desirable to provide a pumping arrangement that enhances the overall efficiency of the pumping system.

### SUMMARY OF THE DISCLOSURE

A fluid filtration system is disclosed, including a fluid storage vessel, a filter housing having first and second ends, a pump coupled between the fluid storage vessel and the filter housing, and a plurality of valves disposed between the pump and the filter housing. The pump is configured to move fluid from the fluid storage vessel through the filter element. The plurality of valves are configurable to selectively direct fluid received from the pump to the first or second end of the filter housing, and to return the fluid to the fluid storage vessel.

The system can also include a first filtration line coupled between the pump and a first end of the filter housing, a second filtration line coupled between the pump and a second end of the filter housing, a first return line coupled between the first end of the filter housing and a common return line, and a second return line coupled between the second end of the filter housing and a common return line. First, second, third, and fourth isolation valves can be disposed, respectively, in the first filtration line, the second filtration line, the first return line, and the second return line, return line, where the first, second, third, and fourth isolation valves can be configured to selectively permit fluid flow between the filter housing and the fluid storage vessel in first and second opposed directions. A common return line may be coupled between the first and second return line and the fluid storage vessel.

A fluid filtration system is disclosed, including a fluid storage vessel, a filter housing having first and second ends, a pump coupled between the fluid storage vessel and the filter housing, a valve assembly comprising a plurality of pinch valves, and a flexible tubing assembly coupled to the valve assembly, the flexible tubing assembly having a plurality of branches coupling the filter housing and the fluid storage vessel. The pump can be configured to move fluid from the fluid storage vessel through the filter element. The plurality of pinch valves may be configurable to selectively direct fluid received from the pump to the first or second end of the filter housing, and to return the fluid from the filter housing to the fluid storage vessel.

The plurality of pinch valves can include first, second, third, fourth, and fifth pinch valves, and the flexible tubing assembly can include a loop contained within the valve assembly and connected to a first branch, a second branch, a third branch, and a fourth branch. The second and fourth branches can be fluidically connected to one another across the loop portion via a connector segment. The first, second, third, and fourth branches can be coupled to respective pump discharge line, first and second filtration lines, and a return line. First and second filtration lines can be coupled between the valve assembly and the filter housing, and the return line can be coupled between the valve assembly and the fluid storage vessel.

A fluid filtration system includes a fluid storage vessel, a first filter housing having first and second ends, a second filter housing having first and second ends, a first pump coupled between the fluid storage vessel and a shuttle valve, and a second pump coupled between the fluid storage vessel and the shuttle valve. The first and second pumps can be configured to move fluid from the fluid storage vessel through the shuttle valve, and through the first and second filter housings. The shuttle valve can be configured for selectively directing fluid received from the first pump and directing the received flow to the first or second end of the first filter housing. The shuttle valve can be configured for selectively directing fluid received from the second pump and directing the received flow to the first or second end of the second filter housing.

The shuttle valve can be configured for receiving fluid from the first or second end of the filter housing and return the received fluid to the fluid storage vessel. In a first mode of operation the shuttle valve can direct flow from the first pump to a first end of the first filter housing and direct flow from the second pump to a second end of the second filter housing. In a second mode of operation the shuttle valve can direct flow from the first pump to a second end of the first filter housing and can direct flow from the second pump to a first end of the second filter housing.

A fluid filtration assembly is disclosed, including a filter housing having a first end connected to a fluid storage vessel, and a syringe pump coupled to a second end of the filter housing. The syringe pump can include a housing portion and a plunger portion, the plunger portion having first end coupleable to an actuator, and a second end having a seal portion for sealing against an inner surface of the housing portion. The plunger portion can be reciprocally movable within housing portion to move fluid between the fluid storage vessel and the filter housing. Moving the plunger portion from a first position to a second position can cause fluid in the housing of the syringe pump to be moved toward the filter housing and causes fluid in the filter housing to move toward the fluid storage vessel. Moving the plunger portion from the second position to the first position can cause fluid in the fluid storage vessel to be moved toward the filter housing and causes fluid in the filter housing to move toward the housing of the syringe pump.

One or more of the disclosed systems can include a flow sensor for determining actual flowrate from a discharge portion of the pump(s). One or more of the disclosed systems can include a controller coupled to the pump(s), the flow sensor, and the valve(s) to selectively control a fluid flow path through the system. One or more of the disclosed systems can include memory associated with the controller, the memory storing a plurality of preset positions of the valve(s). The controller can be programmed to adjust a speed of the pump(s) based on sensed flowrate information received from the flow sensor.

The pump(s) of one or more of the fluid filtration systems can be a low shear pump(s) and a filter element disposed in the filter housing(s) comprise a hollow fiber filter. The fluid storage vessel of one or more of the fluid filtration systems can be a bioreactor. The fluid managed by one or more of the fluid filtration systems can be a cell culture.

In some embodiments the pump(s) can be low shear pump(s). In some embodiments the filter element(s) are hollow fiber filter(s). In some embodiments the fluid storage vessel is a bioreactor. In some embodiments the fluid comprises cell cultures. The cell culture can be a fed-batch cell culture or a concentrated fed-batch cell culture, and the disclosed systems and methods can be used to produce any of a variety of desired cell products including but not limited to endogenous and recombinant products, including proteins, peptides, nucleic acids, virus, amino acids, antibiotics, specialty chemicals and other molecules of value. Desired proteins may include but are not limited to monoclonal antibodies, enzymes and other recombinant antibodies, enzymes, peptides, virus.

### BRIEF DESCRIPTION OF THE DRAWINGS

The accompanying drawings illustrate preferred embodiments of the disclosed method so far devised for the practical application of the principles thereof, and in which:

FIGS. 1A and 1B are schematic views of an example pump and filter system according to the present disclosure operating in a first flow mode;

FIG. 2 is a schematic view of another example pump and filter system according to the present disclosure.

FIG. 3 is an example valve block assembly for use in the system of FIG. 2.

FIG. 4 is an example flexible conduit arrangement for use with the valve block assembly of FIG. 3.

FIGS. 5A and 5B are schematic views of another example pump and filter system according to the present disclosure.

FIG. 6 is a schematic view of another example pump and filter system according to the present disclosure.

### DESCRIPTION OF EMBODIMENTS

A system is disclosed, comprising a bioreactor, a pump and a filter. The pump moves fluid in alternating directions through the filter via associated piping and a flow diverter such as, but not limited to, a three-way valve, a rotary valve, a pinch valve, or a shuttle valve. The system can be employed for conducting a rapid, low sheer, Tangential Flow Filtration (TFF) or Alternating Tangential Flow (ATF) of fluid through the filter, which in some embodiments is a hollow fiber filter. Such a system has applications in perfusion of cultured animal cells as well as other varied filtration applications.

As will be discussed in greater detail later, the disclosed assembly can reduce the amount of time cell cultures reside outside the bioreactor and can also provide a more uniform use of the filter as well as reduced fouling, compared to current systems. In some embodiments, operational control of the pump can be based on an algorithm which can periodically apply an operational subroutine that facilitates a filter cleaning/backflush function. These and other advantage will be discussed below.

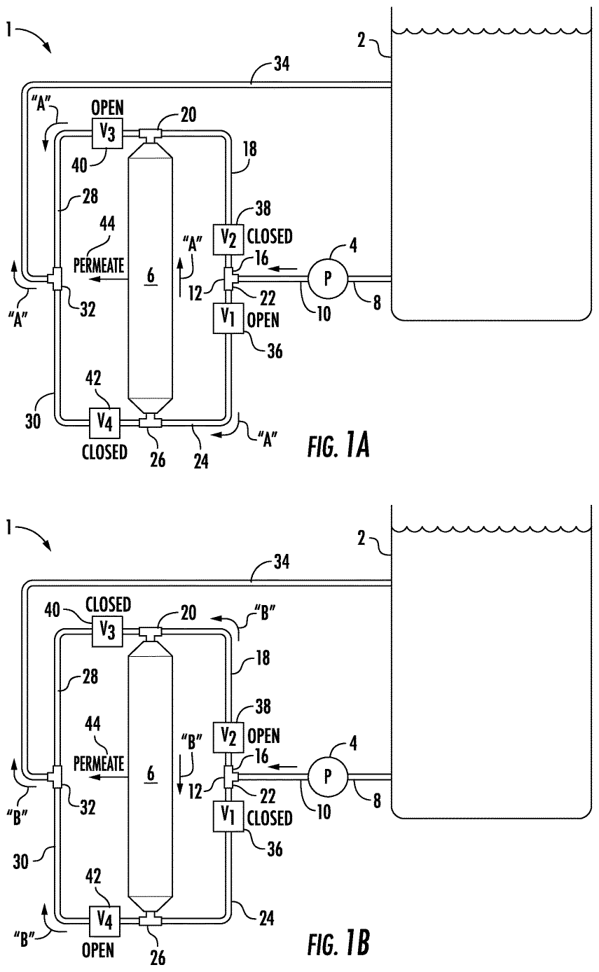

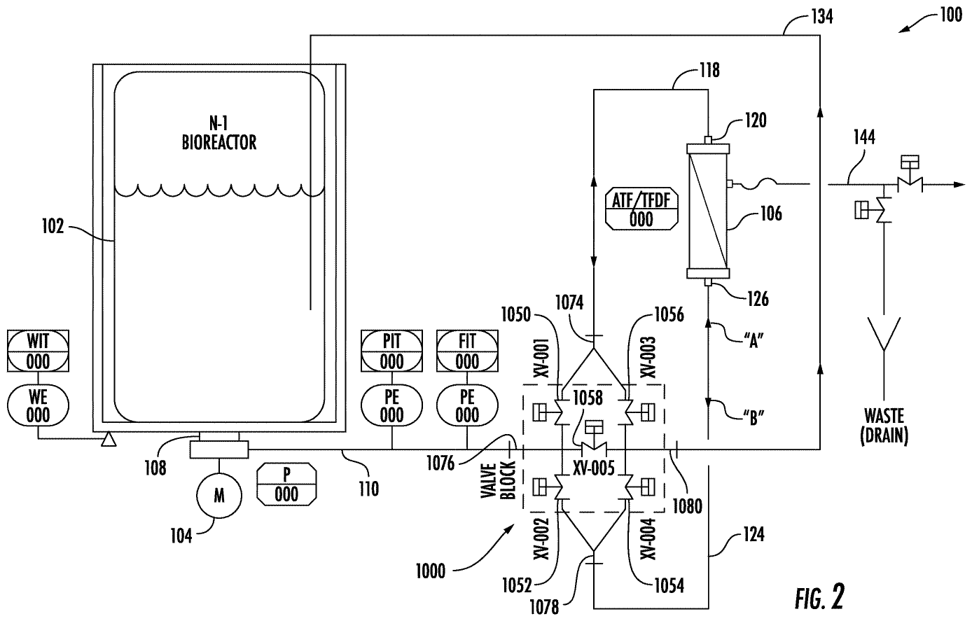

FIGS. 1A and 1B illustrate an example system 1 which can include a fluid storage vessel 2 (referred to herein as “vessel”) coupled to a pump 4 and a filter housing 6. The pump 4 is arranged to take suction from the vessel 2 via a suction line 8 disposed at or near the bottom of the vessel 2. The pump 4 is coupled to a discharge line 10, which in turn is coupled to a first tee connection 12. A first outlet 16 of the tee 12 is coupled to a first filtration line 18, which in turn is coupled to a first end 20 of the filter housing 6. A second outlet 22 of the first tee connection 12 is coupled to a second filtration line 24, which in turn is coupled to a second end 26 of the filter housing 6. A first return line 28 is coupled to the first end 20 of the filter housing 6, while a second return line 30 is coupled to the second end 26 of the filter housing 6. The first and second return lines 28, 30 connect to a second tee connection 32, which is also connected to a common return line 34 which runs from the second tee connection 32 to the vessel 2.

A plurality of valves can be disposed between the vessel 2 and the filter housing 6 for selectively enabling flow between the vessel filter housing. For example, a first isolation valve 36 can be disposed in the second filtration line 24 between the first tee connection 12 and the second end 26 of the filter housing 6. A second isolation valve 38 can be disposed in the first filtration line 18 between the first tee connection 12 and the second end of the filter housing 26. A third isolation valve 40 can be disposed in the first return line 28 between the first end 20 of the filter housing 6 and the second tee connection 32. A fourth isolation valve 42 can be disposed in the second return line 30 between the second end 26 of the filter housing 6 and the second tee connection 32.

Thus, to effect flow through the filter housing 6 (and filter) in the direction of arrow “A” (i.e., the first mode of operation, in which the system 1 is configured to direct flow from the vessel 2 to the second end 26 of the filter housing 6 via the second filtration line 24, the first flow diverter isolation valve 36 is configured in the open position while the second flow diverter isolation valve 38 is configured in the closed position. The third isolation valve 40 is configured in the open position while the fourth isolation valve 42 is configured in the closed position. Thus configured, flow from the pump 4 is directed to the second filtration line 24. The second isolation valve 38, being in the closed position, prevents flow through the first filtration line 18. Fluid thus flows from the vessel 2, through the pump 4, through the first flow diverter isolation valve 36, and through the second filtration line 24 where it enters the second end 26 of the filter housing 6. Fluid travels through the filter housing 6 in the direction of arrow “A” and exits the first end 20 of the filter housing 6. Fluid is filtered within the filter housing 6 and a portion of the fluid (e.g., permeate) can be evacuated from the filter housing 6 via permeate discharge line 44 using a separate pump (not shown). The remaining portion of fluid is returned in a direction toward the vessel 2 via the first return line 28, the second tee 32, and the common return line 34.

To effect flow through the filter housing 6 (and filter) in the direction of arrow “B” (i.e., the second mode of operation, in which the system 1 is configured to direct flow from the vessel 2 to the first end 20 of the filter housing 6 via first filtration line 18), the first isolation valve 36 is configured in the closed position while the second isolation valve 38 is configured in the open position. The third isolation valve 40 is configured in the closed position while the fourth isolation valve 42 is configured in the open position. Thus configured, flow from the pump 4 is directed to the first filtration line 18. The first isolation valve 36, being in the closed position, prevents flow through the second filtration line 24. Fluid thus flows from the vessel 2, through the pump 4, through the second isolation valve 38, and through the first filtration line 18 where it enters the first end 20 of the filter housing 6. Fluid travels through the filter housing 6 in the direction of arrow “B” and exits the second end 26 of the filter housing 6. Fluid is filtered within the filter housing 6 and a portion of the fluid (e.g., permeate) can be evacuated from the filter housing 6 via permeate discharge line 4 using a separate pump (not shown). The remaining portion of fluid is returned in a direction toward the vessel 2 via the second return line 30, the second tee 32, and the common return line 34.

With the embodiment of FIGS. 1A and 1B, a single pump 4 can be used to continuously pump cell culture from the vessel 2 (bioreactor) based on a user-defined flow rate. The cell culture flows through the filter housing 6 and filter element in a single direction (First mode “A” or Second mode “B”) or selectively in two opposing directions (First Mode “A” and Second Mode “B”) based on a selected operation mode. As can be seen, in the embodiment of FIGS. 1A and 1B, flow though the filtration and return lines 18, 24, 28, 30 is unidirectional, and no filtration or return line is exposed to bidirectional flow. The disclosed arrangement may have advantages in reducing pressure/flow fluctuations when flow is alternatingly directed to the first and second ends of the filter housing, as compared to more severe changes in momentum that are inherent in reverse flow regimes of conventional systems.

In an alternating tangential flow mode, cell culture flow can be re-directed by the opening and closing of the first, second, third and fourth isolation valves 36, 38, 40, 42. In addition, where the system 1 is configured to accommodate alternating tangential flow, the pump 4 may not displace the entire volume of cell culture from the vessel 2. Rather, multiple pump cycles may be required to move the entire volume of cell culture through the filter housing 6. Time based pumping commanded by flow of the proposed technology guaranies full exchange of cell culture in one recirculation loop cycle.

The time of flow one direction is the time when a single particulate (cell) travels from the vessel 2 (e.g., bioreactor) through the entire loop of piping (tubing), through the filter 6, and returns to the vessel. This time depends on the flow rate set by the pump 4. An algorithm performed by a controller (not shown) can calculate the time required for the cells to make the complete loop. The same time is than applied to flow of cells the opposite direction. When the pump 4 is set to run at a low flowrate, it will take relatively longer for the cells to make a complete loop in one direction, while setting the pump to run at a higher flowrate will result in a shorter time for the cells to make a complete loop in one direction. The disclosed alternating flow arrangement (i.e., flow through the loop in a first direction follow by flow through the loop in a second, opposite, direction), can be effective in preventing premature filter clogging. Flow through the permeate discharge line 44 can be adjusted by adjusting the speed of the associated permeate pump (not shown) and may be based on the flow rate of a retentate pump (not shown). Retentate and permeate pump flow rate can be periodically increased for a short time period to further help with the cleaning of the filter and to increase its life.

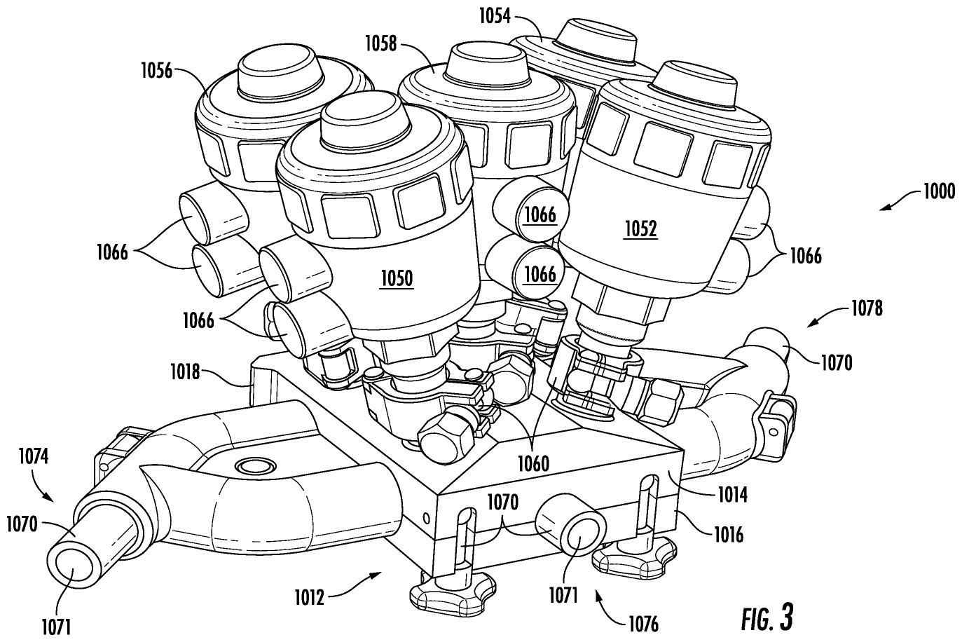

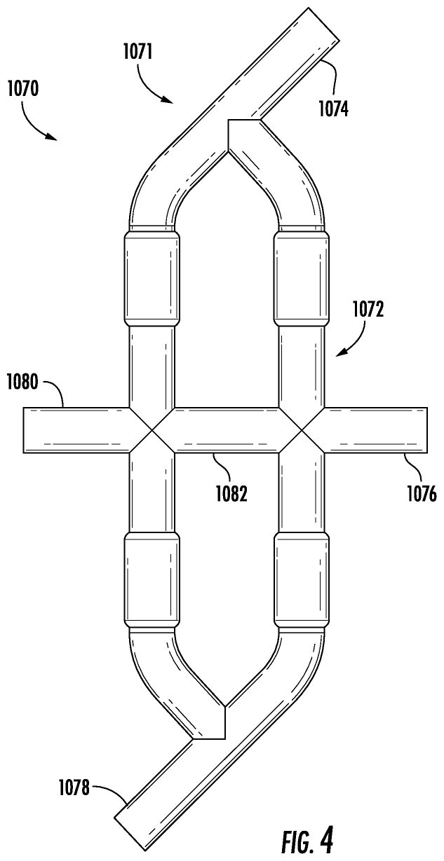

FIG. 2 illustrates an alternative embodiment of the disclosed system 100 in which a flow diverter comprising a valve assembly 1000 (FIG. 3) that encapsulates a flexible tubing arrangement (FIG. 4) for selectively directing flow of process fluid through a filter housing 106 in a single direction (First Mode arrow “A” or Second Mode arrow “B”) or selectively in two opposing directions (First Mode arrow “A” and Second Mode arrow “B”).

The system 100 includes a vessel 102 coupled to a pump 104 and a filter housing 106. The pump 104 is arranged to take suction from the vessel 102 via a suction line 108 disposed at or near the bottom of the vessel 102. The pump 104 is coupled to a discharge line 110, which in turn is coupled to the valve assembly 1000. A first branch 1074 of the valve assembly 1000 is coupled to a first filtration line 118 which in turn is coupled to a first end 120 of the filter housing 106. A third branch 1078 of the valve assembly 1000 is coupled to a second filtration line 120 which in turn is coupled to a second end 126 of the filter housing 106.

FIG. 3 illustrates a non-limiting example embodiment of a valve assembly 1000 that is used to create a multidirectional flow path in the system of FIG. 2. The valve assembly 1000 includes a valve body 1012 that includes a first body portion 1014 and second body portion 1016 that are connected to one another via one or more hinges 1018. The first and second body portions 1014, 1016 when in a closed state, define passageways 1020 that extend through the closed valve body 1012 that receives the flexible conduit 1070. In one embodiment the passageways 1020 are substantially circular in cross section when the valve body 1012 is in the closed state. For example, the first body portion 1014 may include a semi-annular or semi-circular passageway formed in a surface thereof that mates with a corresponding semi-annular or semi-circular passageway formed in a surface of the second body portion 1016 to create the annular or circular passageway 1020. The illustrated layout of the passageways 1020 are formed to accommodate a flexible tubing or conduit 1070 that is placed therein. The flexible tubing or conduit 1070, as disclosed below in more detail below, includes a loop portion 1072 that is utilized to create a multidirectional flow path when used in combination with a series of valves as described herein. The flexible conduit 1070 includes an internal lumen 1071 (see FIG. 3) through which fluid travels.

As can be seen in FIG. 4, the flexible conduit 1070 used in this embodiment includes a loop portion 1072 that defines a circular fluid pathway. Fluidly connected to the loop portion 1072, in this embodiment, are a first branch 1074, a second branch 1076, a third branch 1078, and fourth branch 1080. The flexible conduit 1070 includes a connector segment 1082 that fluidically connects opposing sides of the loop portion 1072. The connector segment 1082 is used to form a bypass pathway between branches 1076, 1078. This connector segment 1082 may be located between the second branch 1076 and the fourth branch 1080 but it also may connect indirectly with branches 1076, 1080 via the loop. The key requirement is that the connector segment 1082 be located in the loop portion 1072 that is inside the closure points described below. This bypass capability provides the ability bypass the filter housing 106 in the event that the filter element disposed within the filter housing fouls during fluid processing, thus providing a methodology of replacing the filter element without shutting down the process. Additionally, operating in bypass mode can provide separate passive mixing option for the vessel 102.

The flexible conduit 1070 is used to carry a fluid through the valve assembly 1000, and may be formed from silicone (e.g., platinum cured silicone), a polymer such as thermoplastic elastomers (TPE), thermoplastic rubber (TPR), or the like. The flexible conduit 1070 may be unreinforced as illustrated or in some embodiments it may be reinforced.

The ends of the branches 1074, 1076, 1078, and 1080 may terminate in a flange or the like such that another segment (not illustrated) may be coupled to the end of the branch 1074, 1076, 1078, and 1080 using a connector or the like.

Referring again to FIG. 2, in the illustrated embodiment, there are five (5) separate valves 1050, 1052, 1054, 1056, 1058 that are secured to the first valve body portion 1014 of the valve body 1012. These valves are referred to as first valve 1050, second valve 1052, third valve 1054, fourth valve 1056, and fifth valve 1058 As explained herein, the valves 1050, 1052, 1054, 1056, 1058 are used to selectively pinch the flexible conduit 1070 that is contained within the valve body 1012. In the illustrated embodiment, each valve 1050, 1052, 1054, 1056, 1058 is secured to the valve body 1012 using a clamp 1060, although the clamp 1060 is optional and in some other embodiments, the valves 1050, 1052, 1054, 1056, 1058 may be secured to the valve body 1012 directly.

Each valve 1050, 1052, 1054, 1056, 1058 includes an actuator (not shown) that includes a pinching element (not shown) to selectively close/open the central lumen 1071 of the flexible conduit 1070. The actuator may be moved using any number approaches. For example, the actuator may be pneumatically actuated valves using air ports 1066 (connected to air lines; not shown). The actuator may also be manually advanced/retracted using a bonnet or the like that is rotated. The actuator may also be actuated using a manually-activated toggle-type mechanism that does not require rotation of a bonnet or the like. This enables one to rapidly switch the valve between on/off states. The actuator may also be actuated with an electrically driven motor or servo. Typically, the valves 1050, 1052, 1054, 1056, 1058 are automatically controlled using off-valve electronics to control the opening/closing states. For example, a solenoid located in a separate control panel assembly (not shown) is used to control air flow to the air ports 1066 to turn on/off valves 1050, 1052, 1054, 1056, 1058.

A first branch 1074 of a flexible conduit 1070 (FIG. 4) is connected to the first filtration line 118, which as mentioned is coupled to the first end 120 of the filter housing 106. A third branch 1078 of the flexible conduit 1070 is connected to the second filtration line 124, which as mentioned is coupled to the second end 126 of the filter housing 106. Fluid from the vessel 102 discharged from pump 104 (e.g., feed pump and upstream instrumentation) enters the valve assembly 1000 via the second branch 1076 of the flexible conduit 1070 and exits the valve assembly 1000 via the fourth branch 1080 of the flexible conduit 1070. In some embodiments the valves are solenoid operated pinch valves.

To effect flow through the filter housing 106 (and filter) in the direction of arrow “A” (i.e., the first mode of operation) in which the system 100 is configured to direct flow from the vessel 102 to the second end 126 of the filter housing 106 via the second filtration line 124), valves 1050, 1054 and 1058 are configured in the closed position, while valves 1052 and 1056 are configured in the open position. Thus configured, fluid flows from the vessel 102, through the pump 104, through valve 1052, then through the second filtration line 124 where it enters the second end 126 of the filter housing 106. Fluid travels through the filter housing 106 in the direction of arrow “A” and exits the first end 120 of the filter housing. Fluid is filtered within the filter housing 106 and a portion of the fluid (e.g., permeate) can be evacuated from the filter housing 106 via permeate discharge line 144 using a separate pump (not shown). The remaining portion of fluid is returned in the direction toward the vessel 102 via the first filtration line 118, valve 1056, and return line 134.

To effect flow through the filter housing 106 (and filter) in the direction of arrow “B” (i.e., the second mode of operation, shown in FIG. 2, in which the system 100 is configured to direct flow from the vessel 102 to the first end 120 of the filter housing 106 via first filtration line 118), valves 1050 and 1054 are configured in the open position, while valves 1052, 1056, and 1058 are configured in the closed position. Thus configured, fluid flows from the vessel 102, through the pump 104, through valve 1050, then through the first filtration line 118 where it enters the first end 120 of the filter housing 106. Fluid travels through the filter housing 106 in the direction of arrow “B” and exits the second end 126 of the filter housing. Fluid is filtered within the filter housing 106 and a portion of the fluid (e.g., permeate) can be evacuated from the filter housing 106 via permeate discharge line 144 using a separate pump (not shown). The remaining portion of fluid is returned in a direction toward the vessel 102 via the second filtration line 124, valve 1054.

With the embodiment of FIGS. 2-4, a single pump 104 can be used to continuously pump cell culture from the vessel 102 (bioreactor) based on a user-defined flow rate. The cell culture flows through the filter housing 106 and filter element in a single direction (First mode “A” or Second mode “B”) or selectively in two opposing directions (First Mode “A” and Second Mode “B”) based on a selected operation mode.

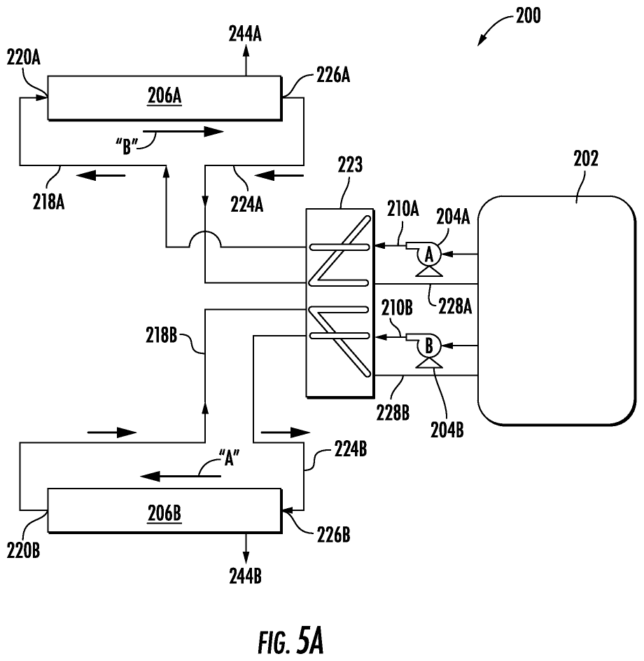

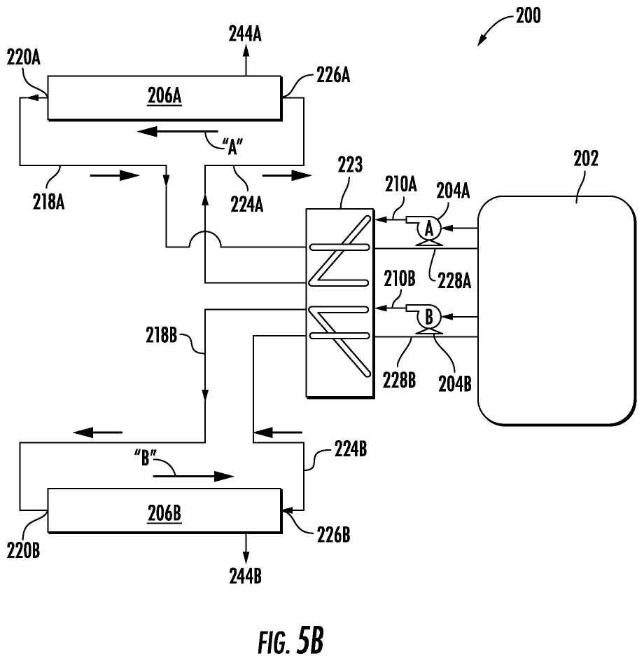

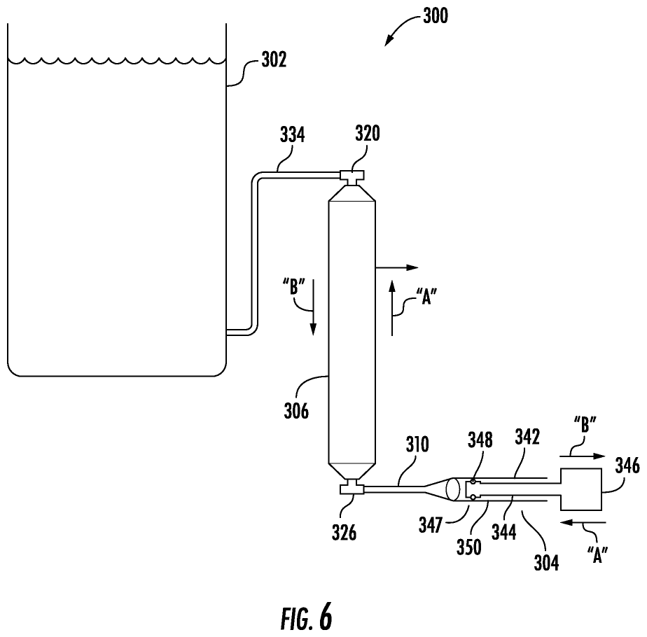

FIGS. 5A and 5B illustrate an alternative embodiment of a disclosed system 200 in which first and second pumps 204A, 204B are employed to direct flow from the vessel 202 to first and second ends 220A, B, 226A, B of first and second filter housings 206A, 206B. A shuttle valve 223 is disposed between the first and second pumps 204A, 204B, and the first and second ends 220A, B, 226A, B of the first and second filter housings 206A, B. The shuttle valve 223 is configured to selectively direct flow from the discharge 210A, 210B of the first and second pumps 204A, 204B to the first end 220A, B or the second end 226A, B of the first and second filter housings 206A, B. The system 200 of FIGS. 5A and 5B, also includes first and second return lines 228A, B coupled between the shuttle valve 223, and the vessel 202. First filtration lines 218A, B are coupled between the shuttle valve 223 and the first ends 220A, 220B of the first and second filter housings 206A, B. Second filtration lines 224A, B are coupled between the 218A, B and 224A, B are coupled between the shuttle valve 223 and the second ends 226A. 226B of the first and second filter housings 206A, B.

To effect flow through the first and second filter housings 206A, B (and filters) in the directions of arrows “A” and “B” (i.e., the first mode of operation, illustrated in FIG. 5A), the shuttle valve 223 is positioned to direct flow from the discharge 210A of the first pump 204A to the first filtration line 218A and to direct flow from the discharge 210B of the second pump 204B to the second filtration line 224B. Thus configured, fluid flows from the vessel 202, through the first pump 204A through the shuttle valve 223, then through the first filtration line 218A where it enters the first end 220A of the first filter housing 206A. Fluid travels through the first filter housing 206A in the direction of arrow “B” and exits the second end 226A of the first filter housing. Fluid is filtered within the first filter housing 206A and a portion of the fluid (e.g., permeate) can be evacuated from the first filter housing 206A via first permeate discharge line 244A using a separate pump (not shown). The remaining portion of fluid is returned in a direction toward the vessel 202 via the second filtration line 224A, shuttle valve 223, and first return line 228A. At the same time, fluid flows from the vessel 202, through the second pump 204B through the shuttle valve 223, then through the second filtration line 224B where it enters the second end 226B of the second filter housing 206B. Fluid travels through the second filter housing 206B in the direction of arrow “A” and exits the first end 220B of the second filter housing. Fluid is filtered within the second filter housing 206B and a portion of the fluid (e.g., permeate) can be evacuated from the second filter housing 206B via second permeate discharge line 244B using a separate pump (not shown). The remaining portion of fluid is returned in a direction toward the vessel 202 via the first filtration line 218B, shuttle valve 223, and second return line 228B.

To effect flow through the first and second filter housings 206A, B (and filters) in the directions of arrows “A” and “B” (i.e., the second mode of operation, illustrated in FIG. 5B), the shuttle valve 223 is positioned to direct flow from the discharge 210A of the first pump 204A to the second filtration line 224A and to direct flow from the discharge 210B of the second pump 204B to the first filtration line 218B. Thus configured, fluid flows from the vessel 202, through the first pump 204A through the shuttle valve 223, then through the second filtration line 224A where it enters the second end 226A of the first filter housing 206A. Fluid travels through the first filter housing 206A in the direction of arrow “A” and exits the first end 220A of the first filter housing. Fluid is filtered within the first filter housing 206A and a portion of the fluid (e.g., permeate) can be evacuated from the first filter housing 206A via first permeate discharge line 244A using a separate pump (not shown). The remaining portion of fluid is returned in a direction toward the vessel 202 via the first filtration line 218A, shuttle valve 223, and first return line 228A. At the same time, fluid flows from the vessel 202, through the second pump 204B through the shuttle valve 223, then through the first filtration line 218B where it enters the first end 220B of the second filter housing 206B. Fluid travels through the second filter housing 206B in the direction of arrow “B” and exits the second end 226B of the second filter housing. Fluid is filtered within the second filter housing 206B and a portion of the fluid (e.g., permeate) can be evacuated from the second filter housing 206B via second permeate discharge line 244B using a separate pump (not shown). The remaining portion of fluid is returned in a direction toward the vessel 202 via the second filtration line 224B, shuttle valve 223, and second return line 228B.

With the embodiment of FIGS. 5A and 5B, first and second pumps 140A, 140B can be used to continuously pump cell culture from the vessel 2 (bioreactor) based on a user-defined flow rate. The cell culture flows through the filter housings 206A, B and filter elements in a single direction or selectively in two opposing directions based on a selected operation mode (e.g., ATF, TFF). The embodiment of FIGS. 5A and 5B also offers instant flow redirection (limited only by the time it takes to move the shuttle of the shuttle valve 223 between the above-described positions).

In the embodiment of FIGS. 5A and 5B, the operation of the first and second pumps 204A, 204B can be continuous, synchronized, according to a timed schedule, and/or manually switched by a user. In a first mode of operation, the first and second pumps 204A, 204B are the same and a flow setpoint of both pumps is the same. The first and second pumps 204A, 204B are arranged for flow to occur in the same direction (i.e., drawing suction from the vessel 202 and discharging toward the filter housings 206A, B).

Referring now to FIG. 6, an alternative embodiment of a disclosed system 300 is illustrated in which a pump 304 is employed to direct flow between a vessel 302 and a filter housing 306. A recirculation line 334 is coupled between the vessel 302 and a first end 320 of the filter housing 306. The pump 304 is coupled via a discharge line 310 to a second end 326 of the filter housing 306. The pump 304 can operate to push and pull process fluid back and forth between the filter housing 306 and the vessel 302. In the illustrated embodiment the pump 304 is a syringe pump having a housing portion 342 and plunger portion 344. The plunger portion 344 may have a first end 346 that can be coupled to an actuation mechanism (not shown), and a second end 347 that has a seal portion 348 that seals against an inner surface 350 of the housing portion 342. Thus arranged, the plunger portion 344 is reciprocally movable with respect to the housing portion 342 in response to activation of the actuator portion. As will be appreciated, a servo motor, cam, pneumatic or electrical actuator can be used to selectively move the plunger portion 344 in the directions of arrows “A” and “B” to cause the plunger portion 344 to move fluid through the filter housing, 306 in a desired manner.

As the actuator moves the first end 346 of the plunger portion 344 in the direction of arrow “A”, the flexible portion forces liquid contained in the housing portion 342 and the discharge line 310 up into the filter housing 306 and back into the vessel 302. In reverse, when the actuator moves the first end 346 of the plunger portion 344 in the direction of arrow “B”, process fluid is drawn from the vessel 302, through the filter housing 306, and into the discharge line 310 and housing portion 342.

In some embodiments, the actuator portion, and thus operation of the pump 304, can be automated via a controller (not shown). The controller may include an appropriate processor and associated memory The processor may execute instructions for actuating the pump 304 according to a desired set of cycle parameters (e.g., stroke distance, stroke rate). The actuator can include a linear encoder (not shown) that can monitor the position of the plunger portion 344 and provide associated position information to the processor and/or other component of the controller. The location of the plunger portion 344 can be monitored throughout an entire actuation cycle of the pump 304. Thus, the controller can monitor the end positions of the plunger portion 344 and can use this information to determine and/or control fluid volume displacement over a particular time period. Due to the mechanical engagement between the actuator and the plunger portion 344, the stroke distance can be known with a relatively high degree of confidence at any point of the actuation process. The disclosed embodiment has the potential for enabling the use of low-cost disposable materials for the plunger and housing, which can lend themselves for use as part of a single use flow path.

It will be appreciated that it in some embodiments it is desirable to minimize the length of the discharge and return lines between the vessel 2, 102, 202, 302 and the filter housing 6, 106, 206, 306 in order to minimize cell culture exposure outside of the vessel.

In some embodiments the filter housing 6, 106, 206, 306 encloses a filter element (not shown), which in one non-limiting exemplary embodiment is a hollow fiber filter, although this is not critical and any of a variety of other filter elements can be used. The filter housing 6, 106, 206, 306 can be made from plastic, metal, such as stainless steel, glass, and the like. Suitable filter elements include hollow fiber filters, screen filters, and the like. In one non-limiting example embodiment, the filter element is a hollow fiber filter. In some implementations, the hollow fiber filter has a pore size of about 0.1 to 5.0 microns, e.g., 0.2, 0.3, 0.4, 0.5, 0.6, 0.7, 0.8, or 0.9 microns, or 1, 2, 3, or 4 microns, or about 500 to 1000 kD, e.g., 550, 600, 650, 700, 750, 800, 850, 900, or 950 kD.

To minimize the negative impact of pumping on the cells within the pumped fluid, a specialized pump type can desirable. In the illustrated embodiments the pumps 4, 104, 204A, 204B can be diaphragm pumps, however, it will be appreciated that the disclosure is not so limited, and thus the pumps 4, 104, 204A, 204B can be any appropriate low-shear pump type, examples of which include Levitronix pumps (www.levitronix.com). Alternatively, a diaphragm pump such as a Quattroflow pump manufactured by Holland Applied Technologies (www.hollandapt.com) may be employed depending on, e.g., shear requirements of an application. Peristaltic pumps can be used for non-cell culture applications. Moreover, permeate and retentate pumps can be peristaltic pumps.

The vessel 2, 102, 202, 302 may be any suitable container for housing a fluid to be filtered. For example, the fluid vessel may be a bioreactor, a fermentor or any other vessel, nonexclusively including vats, barrels, tanks, bottles, flasks, containers, and the like which can contain liquids. The vessel may be composed of any suitable material such as plastic, metal such as stainless steel, glass, or the like.

In one or more embodiments, actuation of the pump 4, 104, 204, 304 and any/all of the disclosed valves can be controlled by a controller to enable the system 1, 100, 200, 300 to operate any or all of the system components in a variety of sequences and manners. The controller may include a processor or microprocessor configured to run an operating system, an application-specific integrated circuit (ASIC), a field-programmable gate array (FPGA), etc., or combinations thereof. The controller may include memory which may include, but is not limited to, electronic, optical, magnetic, or any other storage or transmission device capable of providing a processor, ASIC, FPGA, etc. with program instructions. The memory may include a memory chip, Electrically Erasable Programmable Read-Only Memory (EEPROM), erasable programmable read only memory (EPROM), flash memory, or any other suitable memory from which the controller can read instructions. The instructions may include code from any suitable programming language.

In some embodiments, the processor of the controller may execute instructions (e.g., a subroutine) to actuate some or all of the pumps and/or valves to reconfigure the system between a plurality of operating modes, for example, to cause tangential flow filtration (TFF) and/or alternating tangential flow filtration (ATF) within the filter housing 6, 106, 206, 306. As will be appreciated, the controller may also control the speed of the pump 4, 104, 204, 304 to adjust flow through the system 1, 100, 200, 300. In some embodiments the controller adjusts pump speed based on flow sensed by one or more flow sensors positioned at appropriate locations within the system. A variety of set points and operating positions can be stored in controller memory and executed by the processing portion of the controller upon user command or automatically. The controller can also include a user interface for allowing a user to input information into the controller and/or operate the system in a desired manner.

As will be appreciated the disclosed systems can be used for both tangential flow filtration (TFF) and alternating tangential flow filtration (ATF). For TFF evolutions the system valves (or the pump in the case of the embodiment of FIG. 6) are maintained to direct flow in a single direction through the associated filter housing. For ATF evolutions, the system valves are repositioned periodically (or the pump is operated, in the case of the embodiment of FIG. 6) to alternate flow through the associated filter housing (i.e., alternating between the flow direction of arrow “A” and the flow direction of arrow “B”). As will be appreciated, the disclosed systems can accommodate TFF and ATF operations in a single system. This is an advantage over conventional arrangements which employ separate systems for TFF and ATF.

In various embodiments, a user interface is provided where users of the disclosed systems can input and/or monitor various facets of the system and operation of associated pumps and valves. For example, the user interface may be programmed to display one or more graphical outputs of data received and analyzed by controller. The user interface may also display other data stored in the memory of controller, including type and size of filter, flow direction mode, permeate pump mode (normal, cleaning), TFF mode, ATF mode, system flow, system pressure, and system status (running, off). Further still, additional parameters that may be displayed to the user at the user interface include a flow rate and a cycle time for one or more process steps.

Further still, the user interface of certain exemplary embodiments permits the user to control starting or stopping of a control process carried out by the controller. In some embodiments, starting and stopping functions may be controlled via buttons provided on a touch-screen display, for example. The user interface also allows for input (entry) of specified control parameters.

In some embodiments the filter element is a hollow fiber filter. In some embodiments the fluid storage vessel is a bioreactor. In some embodiments the fluid comprises cell cultures. In some embodiments the flow diverter is selected from the list consisting of a three-way valve, a pinch valve, a rotary valve, and a shuttle valve. In other embodiments the flow diverter comprises first and second flow diverter isolation valves.

As will be understood, the disclosed systems and methods provide a variety of advantages over conventional systems. For example, the disclosed systems and methods use only electrical power for ATF valve system activation (compared to conventional ATF controllers that utilize air pressure and vacuum to activate a diaphragm pump to obtain system flow.) The disclosed systems and methods have applications in perfusion of cultured animal cells as well as other varied filtration applications. Cultured animal cells can mean mammalian cells suspended in a liquid culture medium. Cultured animal cells can have a cell density of greater than about 0.1×10⁶ cells/mL (e.g., greater than about 1×10⁶ cells/mL, greater than about 5×10⁶ cells/mL, greater than about 10x 10⁶ cells/mL, greater than about 15×10⁶ cells/mL, greater than about 20×10⁶ cells/mL, greater than about 25×10⁶ cells/mL, greater than about 30×10⁶ cells/mL, greater than about 35×10⁶ cells/mL, greater than about 40×10⁶ cells/mL, greater than about 45×10⁶ cells/mL, greater than about 50×10⁶ cells/mL, greater than about 55×10⁶ cells/mL, greater than about 60×10⁶ cells/mL, greater than about 65×10⁶ cells/mL, greater than about 70×10⁶ cells/mL, greater than about 75×10⁶ cells/mL, greater than about 80×10⁶ cells/mL, greater than about 85×10⁶ cells/mL, greater than about 90×10⁶ cells/mL, greater than about 95×10⁶ cells/mL, or greater than 100×10⁶ cells/mL).

While the present invention has been disclosed with reference to certain embodiments, numerous modifications, alterations and changes to the described embodiments are possible without departing from the spirit and scope of the invention, as defined in the appended claims. Accordingly, it is intended that the present invention are not limited to the described embodiments, but that it has the full scope defined by the language of the following claims, and equivalents thereof.

1. A fluid filtration system, comprising:
a fluid storage vessel;
a filter housing having first and second ends;
a pump coupled between the fluid storage vessel and the filter housing; and
a plurality of valves disposed between the pump and the filter housing;
wherein the pump is configured to move fluid from the fluid storage vessel through the filter element; and
wherein the plurality of valves are configurable to selectively direct fluid received from the pump to the first or second end of the filter housing, and to return the fluid to the fluid storage vessel.

2. The fluid filtration system of claim 1, further comprising:
a first filtration line coupled between the pump and a first end of the filter housing;
a second filtration line coupled between the pump and a second end of the filter housing;
a first return line coupled between the first end of the filter housing and a common return line;
a second return line coupled between the second end of the filter housing and a common return line; and
first, second, third, and fourth isolation valves disposed, respectively, in the first filtration line, the second filtration line, the first return line, and the second return line, return line, the first, second, third, and fourth isolation valves configured to selectively permit fluid flow between the filter housing and the fluid storage vessel in first and second opposed directions.

3. The fluid filtration system of claim 2, further comprising a common return line coupled between the first and second return line and the fluid storage vessel.

4. A fluid filtration system, comprising:
a fluid storage vessel;
a filter housing having first and second ends;
a pump coupled between the fluid storage vessel and the filter housing;
a valve assembly comprising a plurality of pinch valves; and
a flexible tubing assembly coupled to the valve assembly, the flexible tubing assembly having a plurality of branches coupling the filter housing and the fluid storage vessel;
wherein the pump is configured to move fluid from the fluid storage vessel through the filter element; and
wherein the plurality of pinch valves are configurable to selectively direct fluid received from the pump to the first or second end of the filter housing, and to return the fluid from the filter housing to the fluid storage vessel.

5. The fluid filtration system of claim 4, wherein the plurality of pinch valves comprises first, second, third, fourth, and fifth pinch valves, and wherein the flexible tubing assembly comprises a loop contained within the valve assembly and connected to a first branch, a second branch, a third branch, and a fourth branch, wherein the second and fourth branches are fluidically connected to one another across the loop portion via a connector segment.

6. The fluid filtration system of claim 5, wherein the first, second, third, and fourth branches are coupled to respective pump discharge line, first and second filtration lines, and a return line, wherein first and second filtration lines are coupled between the valve assembly and the filter housing, and the return line is coupled between the valve assembly and the fluid storage vessel.

7. A fluid filtration system, comprising:
a fluid storage vessel;
a first filter housing having first and second ends;
a second filter housing having first and second ends;
a first pump coupled between the fluid storage vessel and a shuttle valve; and
a second pump coupled between the fluid storage vessel and the shuttle valve;
wherein the first and second pumps are configured to move fluid from the fluid storage vessel through the shuttle valve, and through the first and second filter housings;
wherein the shuttle valve is configured for selectively directing fluid received from the first pump and directing the received flow to the first or second end of the first filter housing; and
wherein the shuttle valve is configured for selectively directing fluid received from the second pump and directing the received flow to the first or second end of the second filter housing.

8. The fluid filtration system of claim 7, wherein the shuttle valve is configured for receiving fluid from the first or second end of the filter housing and return the received fluid to the fluid storage vessel.

9. The fluid filtration system of claim 8, wherein in a first mode of operation the shuttle valve directs flow from the first pump to a first end of the first filter housing and directs flow from the second pump to a second end of the second filter housing; and in a second mode of operation the shuttle valve directs flow from the first pump to a second end of the first filter housing and directs flow from the second pump to a first end of the second filter housing.

10-12. (canceled)

13. The fluid filtration system of claim 1, further comprising a flow sensor for determining actual flowrate from a discharge portion of the pump.

14. The fluid filtration system of claim 13, further comprising a controller coupled to the pump, the flow sensor, and the plurality of valves to selectively control a fluid flow path through the system.

15. The fluid filtration system of claim 14, further comprising memory associated with the controller, the memory storing a plurality of preset positions of the plurality of valves

16. The fluid filtration system of claim 13, wherein the controller is programmed to adjust a speed of the pump based on sensed flowrate information received from the flow sensor.

17. The fluid filtration assembly of claim 1, wherein the pump comprises a low shear pump and a filter element is disposed in the filter housing, the filter element comprising a hollow fiber filter.

18. The fluid filtration system of claim 1, wherein the fluid storage vessel is a bioreactor.

19. The fluid filtration system of claim 1, wherein the fluid comprises a cell culture.
